(19) United States
(12) Patent Application Publication
(10) Pub. No.: US 2026/0363888 A1
(43) Pub. Date: Nov. 19, 2026

(54) DELIVERY SYSTEM

(71) Applicant: JAPAN LIFELINE CO., LTD., Tokyo (JP)

(72) Inventors: Masato SERINO, Tokyo (JP); Tomoharu KOISO, Tokyo (JP)

(21) Appl. No.: 19/489,142

(22) PCT Filed: May 9, 2025
(86) PCT No.: PCT/JP2025/017047

§ 371 Date: Dec. 1, 2025

(30) Foreign Application Priority Data
Sep. 13, 2024 (JP) 2024-159065

(51) Int. Cl. A61F 2/966; A61F 2/95

(52) U.S. Cl. CPC A61F 2/966; A61F 2002/9511; A61F 2002/9528

(57) ABSTRACT

A delivery system provided with: an inner catheter; an outer catheter through which the inner catheter is passed and in which a first side hole is formed; a tube stent disposed at a distal end side with respect to the outer catheter, the inner catheter being passed through the tube stent; and a first thread-like member non-releasably connected to the tube stent, at least a portion of the first thread-like member being passed between the inner catheter and the outer catheter at a more proximal end side than the first side hole, and the first thread-like member being passed through the first side hole and being disposed outside the outer catheter in at least a part of an axial range R from the distal end of the outer catheter to the first side hole.

### CROSS REFERENCE TO RELATED APPLICATIONS

This application is the U.S. national stage application under 35 U.S.C. § 371 of International Application No. PCT/JP2025/017047, filed on May 9, 2025, which claims the benefit of and priority to Japanese Patent Application 2024-159065, filed on Sep. 13, 2024, the entire contents of which are incorporated herein by reference.

### TECHNICAL FIELD

The present disclosure relates to a delivery system.

### BACKGROUND

JP 2012-239803 A discloses a delivery system for delivering a tube stent. The delivery system thereof is provided with an inner catheter, an outer catheter through which the inner catheter is passed and in which a first side hole is formed, and a tube stent disposed at a distal end side with respect to the outer catheter, the inner catheter being passed through the tube stent. The delivery system of Patent Literature 1 includes a first thread-like member connected to the tube stent. The first thread-like member is passed between the inner catheter and the outer catheter at both axial sides with respect to the first side hole.

### SUMMARY

When the inner catheter is pulled out toward the proximal end side with respect to the outer catheter, a pull-out resistance is generated in the inner catheter due to friction with the first thread-like member. The inventors of the present application recognized that there is room for improvement in reducing the pull-out resistance of the inner catheter in the delivery system of Patent Literature 1.

Accordingly, one object of the present disclosure is to provide a delivery system that is advantageous for reducing the pull-out resistance of an inner catheter.

A delivery system of the present disclosure is provided with: an inner catheter; an outer catheter through which the inner catheter is passed and in which a first side hole is formed; a tube stent disposed at a distal end side with respect to the outer catheter, the inner catheter being passed through the tube stent; and a first thread-like member non-releasably connected to the tube stent, at least a portion of the first thread-like member being passed between the inner catheter and the outer catheter at a more proximal end side than the first side hole, in which the first thread-like member is passed through the first side hole and is disposed outside the outer catheter in at least a part of an axial range from the distal end of the outer catheter to the first side hole.

The delivery system according to the present disclosure is advantageous for reducing the pull-out resistance of an inner catheter.

### BRIEF DESCRIPTION OF DRAWINGS

FIG. 1 is an explanatory view of a usage example of a tube stent.

FIG. 2 is a side view of a delivery system of a first embodiment.

FIG. 3 is an enlarged schematic view of a portion of FIG. 2.

FIG. 4 is a side cross-sectional view schematically illustrating a first thread-like member of the first embodiment together with surrounding structures.

FIG. 5 is a view of a portion of the delivery system as seen from the direction of arrow V in FIG. 4.

FIG. 6 is a side cross-sectional view schematically illustrating a second thread-like member of the first embodiment together with surrounding structures.

FIG. 7 is an explanatory view schematically showing an operation when a connection by the second thread-like member is released.

FIG. 8 is a side cross-sectional view schematically illustrating a first thread-like member of a second embodiment together with surrounding structures.

FIG. 9 is a schematic side cross-sectional view illustrating a first thread-like member of a third embodiment from the same perspective as FIG. 4.

### DESCRIPTION OF EMBODIMENTS

Hereinafter, embodiments for carrying out the delivery system of the present disclosure will be described. Same or equivalent elements are assigned the same reference numerals, and duplicate descriptions are omitted. In each drawing, constituent elements are omitted, enlarged, or reduced, as appropriate, for convenience of description. Drawings are to be viewed in the orientation of the reference numerals. In the present specification, a notation of “nth” (n being a natural number) such as “first” or “second” is used only as a formal description to distinguish between multiple elements, and does not have any other substantial meaning. For example, each element denoted by “nth” may be present independently without continuity. That is, a “second” element may be present without the presence of a “first” element.

### First Embodiment

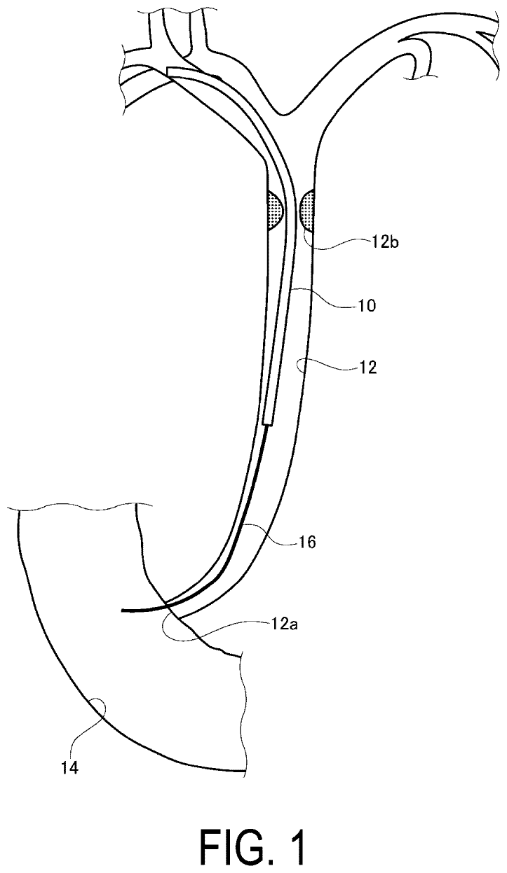

A first embodiment will be described with reference to FIG. 1. First, a usage scenario of a tube stent 10 used in the delivery system will be described. The tube stent 10 is used for the purpose of an action (hereinafter, referred to as a treatment) relating to a medical treatment or examination of a living body. The tube stent 10 of the present embodiment is used for the purpose of drainage, such as bile duct drainage and pancreatic duct drainage.

The living body includes a first organ 12 to be treated and a second organ 14 into which an entrance 12a of the first organ 12 opens. Here, an example in which the first organ 12 is a bile duct and the second organ 14 is a duodenum will be described. In a case in which the tube stent 10 is used for the purpose of drainage, the bile duct or the like serving as the first organ 12 has a stricture 12b that inhibits the discharge of bodily fluids such as bile. By placing the tube stent 10 at the location of the stricture 12b in the first organ 12, discharge of bodily fluid through the tube stent 10 can be promoted.

The tube stent 10 of the present embodiment is used as an inside stent that is entirely disposed more inward in the first organ 12 than the entrance 12a. In this case, when the tube stent 10 is placed inside the first organ 12, a first thread-like member 16 connected to the tube stent 10 is also placed inside the body. A part of the first thread-like member 16 is pulled out from the entrance 12a of the first organ 12 and positioned in the second organ 14. The tube stent 10 in the first organ 12 can be removed by pulling a part of the first thread-like member 16 located in the second organ 14.

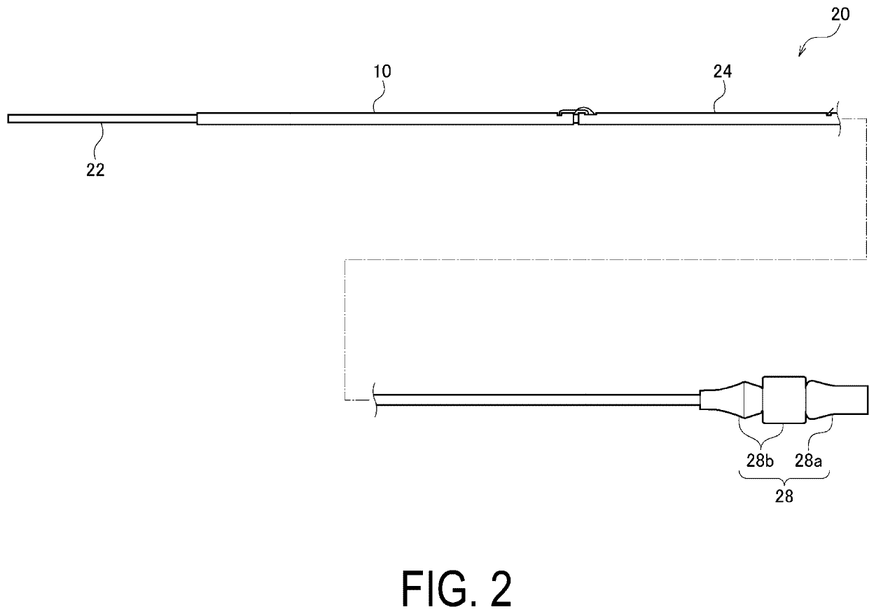

The present disclosure will be further described with reference to FIG. 2. A delivery system 20 in which the tube stent 10 is used will be described. The delivery system is used to deliver the tube stent 10 to the first organ 12 (in the present embodiment, the stricture 12b of the first organ 12) to be treated.

The delivery system 20 includes, in addition to the tube stent 10 described above, an inner catheter 22, an outer catheter 24 through which the inner catheter 22 is passed, and a connector 28 that releasably connects the inner catheter 22 and the outer catheter 24. The tube stent 10 is disposed at a distal end side with respect to the outer catheter 24, and the inner catheter 22 is passed therethrough.

Hereinafter, a direction along a center line of the inner catheter 22 is called an axial direction, and a radial direction and a circumferential direction based on the center line are referred to simply as the radial direction and the circumferential direction. In the delivery system 20, the distal end side (the left side in the drawing), which is one end side in the axial direction, is inserted into the body, and the proximal end side (the right side in the drawing), which is the other end side in the axial direction, is disposed outside the body. For convenience, FIG. 2 shows a state in which the tube stent 10 and the inner catheter 22 are linearly extended. These may be partially curved or bent when no external force is applied.

The tube stent 10, the inner catheter 22, and the outer catheter 24 are each a tubular member having flexibility that allows the member to bend and deform. A guidewire lumen (not shown) is formed inside the inner catheter 22. The connector 28 includes a first connector part 28a attached to the proximal end portion of the inner catheter 22, and a second connector part 28b attached to the proximal end portion of the outer catheter 24 and to which the first connector part 28a is releasably connected.

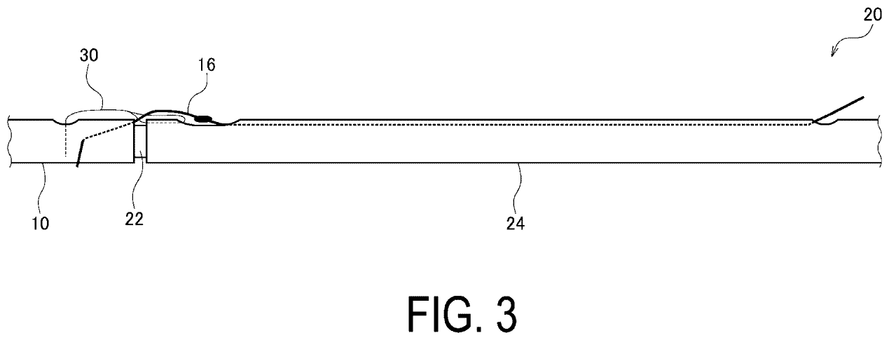

The present disclosure will be further described with reference to FIG. 3. The delivery system 20 is provided with a first thread-like member 16 that is non-releasably connected to the tube stent 10, and a second thread-like member 30 that releasably connects the tube stent 10 and the outer catheter 24. The first thread-like member 16 is not used for the purpose of connecting the tube stent 10 and the outer catheter 24, the connection thereof being carried out by the second thread-like member 30. The role of the second thread-like member 30 is to prevent the outer catheter 24 and the tube stent 10 from being separated from each other in the axial direction. The delivery system 20 of the present embodiment is characterized by the first and second thread-like members 16 and 30 and the surrounding structures related thereto. These will be further described below.

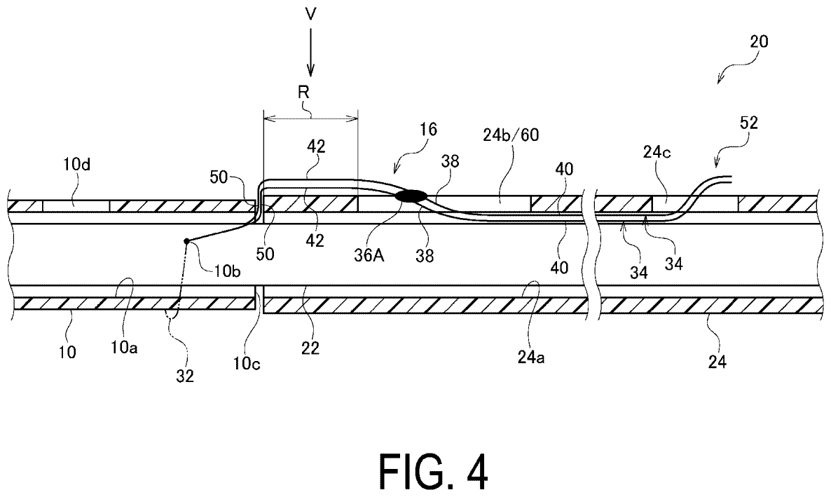

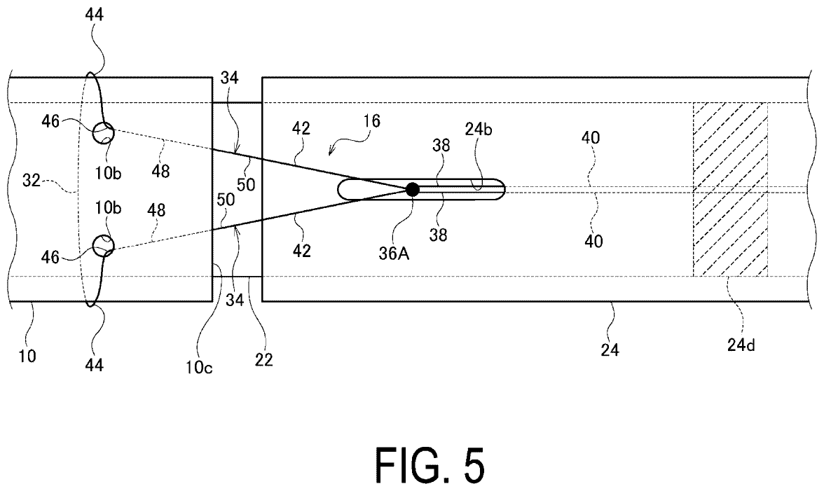

The following description references FIGS. 4 and 5. In FIGS. 4 and 5, the second thread-like member 30 is omitted. A lumen 24a through which the inner catheter 22 passes is formed inside the outer catheter 24. A lumen 10a through which the inner catheter 22 passes is formed inside the tube stent 10.

A first side hole 24b is formed at a distal end portion of the outer catheter 24. The first side hole 24b penetrates the outer catheter 24 in the radial direction at a location separating the lumen 24a from the external space. A second side hole 24c is formed in the outer catheter 24 further to the proximal end side than the first side hole 24b. The second side hole 24c penetrates the outer catheter 24 in the radial direction at a location that separates the first lumen 24a from the external space. The first side hole 24b is provided further to the distal end side than a marker portion 24d provided in the outer catheter 24. The marker portion 24d is formed from a material that is impermeable to radiation such as X-rays. The material is, for example, barium, gold, platinum, or the like. The marker portion 24d is used to specify the position of the distal end portion of the outer catheter 24 in a captured image obtained using radiation. The marker portion 24d of the present embodiment is provided through a ring member separate from the outer catheter 24. In addition, the marker portion 24d may be provided integrally with the outer catheter 24 as a part of the outer catheter 24 by kneading the material that is impermeable to radiation with a part of the outer catheter 24. The marker portion 24d of the present embodiment is provided on an inner circumferential portion of the outer catheter 24 forming the first lumen 24a, but it may be provided on an outer circumferential portion thereof.

As described above, the first thread-like member 16 is non-releasably connected to the tube stent 10. Here, the phrase “non-releasably connected” means that the first thread-like member 16 is not structured to be releasable from a connected state when the first thread-like member 16 is connected to the tube stent 10. As described later, the second thread-like member 30 has a structure in which the connected state between the tube stent 10 and the outer catheter 24 by the second thread-like member 30 can be released by a pulling operation of the inner catheter 22. The phrase “non-releasably connected” in relation to the first thread-like member 16 can also be said to mean that the first thread-like member 16 is not structured such that the connected state of the first thread-like member 16 to the tube stent 10 can be released by an operation of another member, as is the case with the second thread-like member 30. It can also be said that the phrase “non-releasably connected” does not mean that the first thread-like member 16 is hooked on another member, and the hooking on the other member can be released by a pulling operation of pulling the other member toward the proximal end side, whereby the connected state to the tube stent 10 can be released. The phrase “non-releasably connected” is intended to address the question of whether the first thread-like member 16 is structured such that the connected state of the first thread-like member 16 can be released. Therefore, even if the connected state of the first thread-like member 16 to the tube stent 10 can be released by breaking the first thread-like member 16, such a case is not taken into consideration in determining whether the condition of “non-releasably connected” is satisfied. It should be noted that the first thread-like member 16 is not non-releasably connected to the outer catheter 24. Accordingly, when the tube stent 10 is placed in the body, the outer catheter 24 can be separated from the tube stent 10 without being restricted by the first thread-like member 16. It can be said that the first thread-like member 16 is provided so as not to hinder the separation of the outer catheter 24 from the tube stent 10 when the outer catheter 24 is to be separated from the tube stent 10.

The first thread-like member 16 has a fold-back portion 32 connected to the tube stent 10, and a pair of thread-like parts 34 connected to the fold-back portion 32. In the present specification, a description of “has” a specific part (here, the fold-back portion 32 or the like) may also mean that the mentioned target (here, the first thread-like member 16) forms the specific part (here, the fold-back portion 32 or the like).

The fold-back portion 32 of the present embodiment is formed between a pair of thread holes 10b by passing the first thread-like member 16 through the pair of thread holes 10b, which are formed in the tube stent 10, and the fold-back portion 32 is hooked onto the tube stent 10 and thereby connected to the tube stent 10. In FIG. 4, the thread hole 10b is indicated by a black circle, and the part of the first thread-like member 16 located outside the tube stent 10 is indicated by the dotted-dashed line. The fold-back portion 32 of the present embodiment is formed between the pair of thread holes 10b and outside the tube stent 10, and is hooked on the outer circumferential portion of the tube stent 10. In addition, the fold-back portion 32 may be formed between the pair of thread holes 10b and inside the tube stent 10 and hooked on the inner circumferential portion of the tube stent 10 or on the thread holes 10b. In addition, the first thread-like member 16 may be passed through a single thread hole 10b to form the fold-back portion 32 at the portion of the first thread-like member that is passed through the thread hole 10b thereof, and the fold-back portion 32 may then be hooked to the thread hole 10b thereof and thereby connected to the tube stent 10. The single thread hole 10b may be a below-described third side hole 10d of the tube stent 10, or may be formed separately from the third side hole 10d. In either case, the fold-back portion 32 of the present embodiment is formed by passing the first thread-like member 16 through the thread hole 10b of the tube stent 10, and is hooked to the tube stent 10 and thereby connected thereto.

In the case in which the fold-back portion 32 is connected to the tube stent 10 by hooking, if the pair of thread-like parts 34 can freely move relative to each other, the connected state of the first thread-like member 16 to the tube stent 10 could be released by pulling either of the thread-like parts 34 toward the proximal end side. In order to avoid this, the first thread-like member 16 is provided with a coupling part 36A (described later) that couples the pair of thread-like parts 34. Through use of the coupling part 36A, a situation in which the connected state of the first thread-like member 16 is released due to free relative movement of the pair of thread-like parts 34 can be avoided, and the connected state of the first thread-like member 16 can be made non-releasable.

At least a portion of the first thread-like member 16 is passed between the inner catheter 22 and the outer catheter 24 at a more proximal end side than the first side hole 24b. In the present embodiment, the portion satisfying this condition is only the portion of the first thread-like member 16 at a more proximal end side than the first side hole 24b, but it may be the entire first thread-like member 16. In addition, in the present embodiment, this condition is satisfied for each of the pair of thread-like parts 34.

The first thread-like member 16 is passed through the first side hole 24b. An axial range R from the distal end of the outer catheter 24 to the first side hole 24b is examined. At this time, the first thread-like member 16 is disposed outside the outer catheter 24 in at least a part of the axial range R. Thus, the first thread-like member 16 is not disposed inside the outer catheter 24 over the entire axial range R. In the present embodiment, the portion satisfying this condition is the entire first thread-like member 16 in the axial range R, but it may be a portion thereof. In the present embodiment, this condition is satisfied for each of the pair of thread-like parts 34.

The first thread-like member 16 has a first passing portion 38 that is passed through the first side hole 24b, a first inner portion 40 that is continuous with the first passing portion 38, and a first outer portion 42 that is continuous with the first passing portion 38. In the present embodiment, each of the pair of thread-like parts 34 form a first passing portion 38, a first inner portion 40, and a first outer portion 42.

The first inner portion 40 is passed between the inner catheter 22 and the outer catheter 24 at a more proximal end side than the first side hole 24b. The matter of the first inner portion 40 being “continuous with the first passing portion 38” means that an outer portion disposed outside of the outer catheter 24 is not present between the first inner portion 40 and the first passing portion 38.

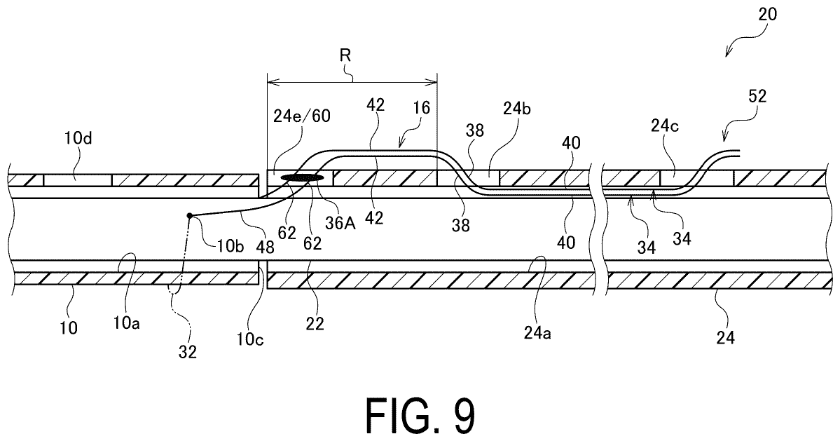

The first outer portion 42 is disposed outside the outer catheter 24 at a more distal end side than the first side hole 24b. The matter of the first outer portion 42 being “continuous with the first passing portion 38” means that an inner portion disposed inside the outer catheter 24 is not present between the first outer portion 42 and the first passing portion 38. The first outer portion 42 of the present embodiment is provided across the entire axial range R described above, but as described below with reference to FIG. 9, it may be provided only in a portion of the axial range R.

A portion of each of the pair of thread-like parts 34 in the present embodiment is disposed inside the tube stent 10. In order to realize this, each of the pair of thread-like parts 34 of the present embodiment has a second outer portion 44 that forms the fold-back portion 32 and is located outside the tube stent 10, a second passing portion 46 that is continuous with the second outer portion 44 and is passed through a thread hole 10b, and a second inner portion 48 that is continuous with the second passing portion 46 and is located inside the tube stent 10. The second inner portion 48 of the present embodiment is drawn out to the proximal end side from an opening 10c formed in the proximal end portion of the tube stent 10 and communicating with the lumen 10a.

The first thread-like member 16 of the present embodiment has an intermediate portion 50 that connects a portion of the outer catheter 24 in a certain axial range with a portion of the tube stent 10 in a certain axial range. The intermediate portion 50 of the present embodiment is formed by each of the pair of thread-like parts 34. The intermediate portion 50 of the present embodiment connects the first outer portion 42 and the second inner portion 48 of the first thread-like member 16. The intermediate portion 50 of the present embodiment is disposed so as to pass between the tube stent 10 and the outer catheter 24.

The coupling part 36A of the present embodiment couples the intermediate portions within a length range from the fold-back portion 32 to the end of each of the pair of thread-like parts 34. The coupling part 36A of the present embodiment is formed by a knot that connects the pair of thread-like parts 34, but specific examples thereof are not particularly limited. In addition, the coupling part 36A may be formed, for example, by an adhered location of the pair of thread-like parts 34 at which an adhesive is used. The coupling part 36A of the present embodiment includes a first coupling part 36A provided at a distal end side portion of the first thread-like member 16.

The first thread-like member 16 has a pull-out portion 52 that is pulled out from the second side hole 24c to the outside of the outer catheter 24. Accordingly, a portion (the pull-out portion 52) of the first thread-like member 16 at a more proximal end side than the first side hole 24b can be grasped from the outside of the outer catheter 24 using a surgeon's fingers, forceps, or the like. The pull-out portion 52 has a portion located inside the second side hole 24c of the outer catheter 24 and a portion located further to the outer side of the outer catheter 24 than the second side hole 24c. The pull-out portion 52 of the present embodiment is formed by each of the pair of thread-like parts 34. The pull-out portion 52 of the present embodiment forms a proximal end portion of the first thread-like member 16.

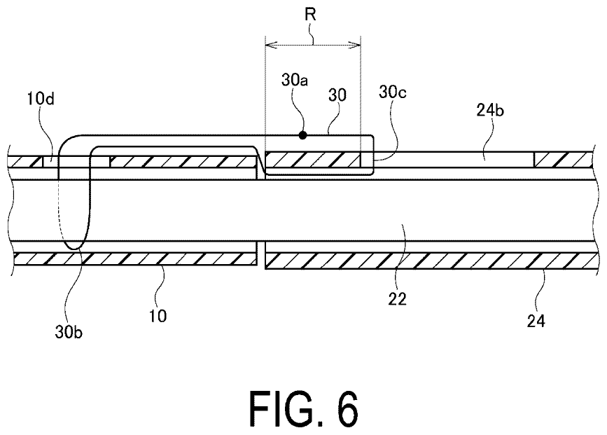

The present disclosure will be further described with reference to FIG. 6. A third side hole 10d is formed in a proximal end side portion of the tube stent 10. The third side hole 10d radially penetrates the tube stent 10 at a location separating the lumen 10a from the external space.

The second thread-like member 30 of the present embodiment includes a coupling part 30a that couples both end portions thereof. The coupling part 30a is formed by a knot that connects both end portions of the second thread-like member 30, an adhered location at which an adhesive is used, or the like.

In the present embodiment, the second thread-like member 30 has a first loop portion 30b wound around the inner catheter 22 and a second loop portion 30c hooked onto the outer catheter 24. The pair of portions forming the first loop portion 30b of the second thread-like member 30 is passed through the third side hole 10d of the tube stent 10. The second loop portion 30c is threaded through the first side hole 24b of the outer catheter 24 such that a portion of the outer catheter 24 is inside the second loop portion 30c. The second loop portion 30c of the second thread-like member 30 is provided so as to pass along the outer side of the outer catheter 24, through the first side hole 24b, and along the inner side of the outer catheter 24 in this order in the axial range R. Accordingly, the outer catheter 24 and the tube stent 10 are connected by the second thread-like member 30.

A case is next considered in which the outer catheter 24 and the tube stent 10 attempt to separate in the axial direction. In this case, the second thread-like member 30 is hooked onto the distal end side portion of the first side hole 24b of the outer catheter 24. In addition, the second thread-like member 30 is wound around the inner catheter 22, and thereby the second thread-like member is prevented from slipping out of the third side hole 10d of the tube stent 10. In this manner, the proximal end side portion of the third side hole 10d is hooked to the second thread-like member 30, which is prevented from slipping out of the third side hole 10d. As a result, axial separation of the outer catheter 24 and the tube stent 10 is prevented.

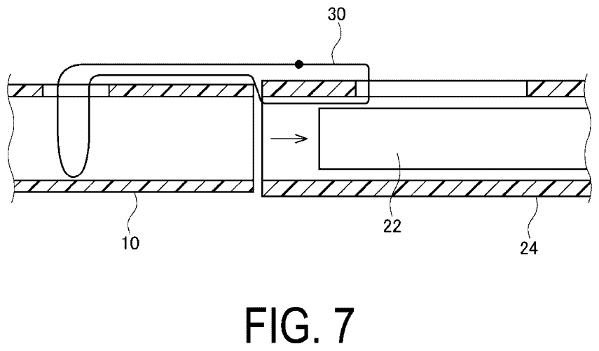

The present disclosure will be further described with reference to FIG. 7. In the present embodiment, the connection between the outer catheter 24 and the tube stent 10 by the second thread-like member 30 can be released by pulling the inner catheter 22 toward the proximal end side of the tube stent 10. This pulling operation is carried out by pulling the inner catheter 22 together with the first connector part 28a of the connector 28 toward the proximal end side. Through this pulling operation, the winding of the second thread-like member 30 around the inner catheter 22 is released, and the connection between the outer catheter 24 and the tube stent 10 by the second thread-like member 30 is released. At this time, the outer catheter 24 is moved together with the second thread-like member 30 toward the proximal end side, and thereby the second thread-like member 30 is pulled out from the third side hole 10d of the tube stent 10, and separation of the outer catheter 24 and the tube stent 10 in the axial direction is allowed.

The tube stent 10 may be formed of various materials including various resin-based materials such as PTFE. The inner catheter 22 may be formed of various materials used for catheters, including various resin-based materials such as olefin-based resins. The outer catheter 24 may be formed of various materials including various resin-based materials such as PTFE. The first and second thread-like members 16 and 30 are formed of, for example, a thread material that uses resin fibers, carbon fibers, metal fibers, or the like. The first and second thread-like members 16 and 30 may be formed of a single thread material or may be formed by twisting a plurality of thread materials.

A usage example of the delivery system 20 explained above will be described. First, prior to use of the delivery system 20, a guidewire is inserted in advance through the second organ 14 and to the first organ 12 to be treated. Next, the guidewire is passed through the guidewire lumen of the inner catheter 22. The delivery system 20 is advanced along the guidewire and through the second organ 14 until the tube stent 10 reaches the target position in the first organ 12. At this time, the delivery system 20 is fed through the lumen of an endoscope.

After the tube stent 10 is disposed at the target position in the first organ 12, the connection between the inner catheter 22 and the outer catheter 24 by the connector 28 is released, and the inner catheter 22 is pulled out from the tube stent 10 and the outer catheter 24. As a result, the connection between the tube stent 10 and the outer catheter 24 by the second thread-like member 30 is released. Subsequently, the outer catheter 24 is pulled out, and thereby the tube stent 10 is placed in the body together with the first thread-like member 16. At this time, as described above, a part of the first thread-like member 16 is placed in the second organ 14.

The effect of the delivery system 20 described above will be described. The first thread-like member 16 is disposed outside the outer catheter 24 in at least a part of an axial range R from the distal end of the outer catheter 24 to the first side hole 24b. Therefore, compared to a case in which the first thread-like member 16 is disposed between the outer catheter 24 and the inner catheter 22 in the entire axial range R, the contact area of the first thread-like member 16 with the inner catheter 22 can be reduced, which is advantageous for reducing the pull-out resistance of the inner catheter 22.

The first thread-like member 16 forms a first outer portion 42 disposed outside the outer catheter 24 at the distal end side of the first side hole 24b. A case in which the first thread-like member 16 is disposed between the outer catheter 24 and the inner catheter 22 over the entire axial range R described above will be considered. According to the present embodiment, compared to such a case, the contact area of the first thread-like member 16 with the inner catheter 22 can be reduced, which is advantageous for reducing the pull-out resistance of the inner catheter 22.

Each of the pair of thread-like parts 34 forms a first outer portion 42 disposed outside the outer catheter 24 at a more distal end side than the first side hole 24b. A case in which the pair of thread-like parts 34 are disposed between the outer catheter 24 and the inner catheter 22 over the entire axial range R described above will be considered. According to the present embodiment, compared to such a case, the contact area of each of the pair of thread-like parts 34 with the inner catheter 22 can be reduced, which is advantageous for reducing the pull-out resistance of the inner catheter 22.

If slack occurs in the first thread-like member 16, the first outer portion 42 of the first thread-like member 16 is likely to interfere with an external object (for example, the endoscope or the like). In regard to this problem, according to the present embodiment, the pull-out portion 52 of the first thread-like member 16 is pulled out to the outside of the outer catheter 24. Therefore, when the delivery system 20 is outside the body, the pull-out portion 52 of the first thread-like member 16 is pulled toward the proximal end side in a state of being pinched by the fingers of the surgeon, forceps, or the like, and thus, slack of the first outer portion 42 of the first thread-like member 16 can be eliminated, and interference with an external object due to the slack can be suppressed.

Next, other features of the delivery system 20 will be described. Description will be made with reference to FIG. 4. The outer catheter 24 includes an accommodation hole 60 that can accommodate the first coupling part 36A. The accommodation hole 60 of the present embodiment is formed by the first side hole 24b of the outer catheter 24. Alternatively, as described below with reference to FIG. 9, the accommodation hole 60 may be formed by the fourth side hole 24e formed in the outer catheter 24 further to the distal end side than the first side hole 24b. In either case, the accommodation hole 60 penetrates the outer catheter 24 in the radial direction at a location that separates the first lumen 24a from the external space. The first coupling part 36A need not always be disposed in the accommodation hole 60 in order to satisfy the condition of “can accommodate” as described herein. For example, if slack has occurred in the first thread-like member 16, the first coupling part 36A may be pulled out from the accommodation hole 60 to the outside of the outer catheter 24. The first coupling part 36A may be accommodated in the accommodation hole 60 when, for example, the proximal end portion of the first thread-like member 16 is pulled toward the proximal end side to apply a tensile force to the entire first thread-like member 16. When the delivery system 20 is fed so as to pass through the lumen of an endoscope, the first coupling part 36A is accommodated in the accommodation hole 60, and thereby an increase in frictional resistance due to contact between the lumen forming surface of the endoscope and the first coupling part 36A can be suppressed.

When accommodated in the accommodation hole 60, the first coupling part 36A is provided in a portion that is continuous with an outer portion disposed outside the outer catheter 24. In the present embodiment, the “outer portion” here is exemplified by the first outer portion 42 described above, but it may be another outer portion provided further to the distal end side than the first outer portion 42. In addition, the “portion that is continuous” as described herein is exemplified in the present embodiment by the first passing portion 38 described above, but it may be a portion that passes through the accommodation hole 60 formed further to the distal end side than the first side hole 24b. An inner portion disposed inside the outer catheter 24 is not present between the outer portion and this portion that is continuous with the outer portion. This advantage will be explained.

A case will be considered in which a portion of the first thread-like member 16 that is continuous with both sides in the length direction with respect to a portion that includes the first coupling part 36A is an inner portion disposed between the outer catheter 24 and the inner catheter 22. In this case, a portion of the first thread-like member 16 abuts against the inner surface of the outer catheter 24 at a location (inner portion) that is continuous with both sides in the length direction with respect to the first coupling part 36A, and thus, radial outward movement of the first coupling part 36A is easily restricted. As a result, when the inner catheter 22 is to be pulled out, a problem occurs in that the state in which the first coupling part 36A is in contact with the inner catheter 22 is easily maintained, and the pull-out resistance of the inner catheter 22 is easily increased due to the first coupling part 36A.

In regard to this problem, according to the present embodiment, when the first coupling part 36A of the first thread-like member 16 is accommodated in the accommodation hole 60, the first coupling part 36A is provided in a portion continuous with the outer portion disposed outside the outer catheter 24. Therefore, with the first thread-like member 16, at a location that is continuous with the outer portion side of the portion where the first coupling part 36A is provided, a portion of the first thread-like member 16 does not abut against the inner surface of the outer catheter 24, and radial outward movement of the first coupling part 36A is facilitated. As a result, when the inner catheter 22 is to be pulled out, the state in which the first coupling part 36A is in contact with the inner catheter 22 is not easily maintained, and an increase in the pull-out resistance of the inner catheter 22 due to the first coupling part 36A can be avoided.

The accommodation hole 60 is set to a size that allows at least the first coupling part 36A of the first thread-like member 16 to pass therethrough. The accommodation hole 60 of the present embodiment is an elongated hole that is long in the axial direction. Accordingly, even if the outer catheter 24 is displaced in the axial direction with respect to the tube stent 10, the first coupling part 36A can be stably accommodated in the accommodation hole 60. The accommodation hole 60 of the present embodiment extends linearly along the axial direction. Alternatively, the accommodation hole 60 may extend obliquely in the circumferential direction toward the axial direction. The minimum width of the accommodation hole 60 in the width direction orthogonal to the length direction is denoted by W. At this time, the length in the length direction of the accommodation hole 60 as an elongated hole is not particularly limited, but may be, for example, twice or more the minimum width W.

The diameter of the first thread-like member 16 may be larger than the diameter of the second thread-like member 30. The diameter herein refers to the maximum diameter (m) in a cross section orthogonal to the length direction of the first thread-like member 16. When the first thread-like member 16 has the coupling part 36A, the diameter of the first thread-like member 16 is the diameter at a location where the coupling part 36A is not present. In order to satisfy such a condition, the diameter of the first thread-like member 16 may be twice or more the diameter of the second thread-like member 30. Through such a design, the strength of the first thread-like member 16 can be better ensured as compared with a case in which the diameter of the first thread-like member 16 is the same as the diameter of the second thread-like member 30. Therefore, such a design is advantageous for preventing breakage of the first thread-like member 16 when the first thread-like member 16 is pulled to remove the tube stent 10 placed in the body. In addition, the second thread-like member 30 can be made less likely to interfere with other constituent elements in comparison to a case in which the diameter of the second thread-like member 30 is the same as the diameter of the first thread-like member 16.

### Second Embodiment

A delivery system 20 of a second embodiment will be described. In the subsequent embodiment, the same details as in the first embodiment may be applied to the constituent elements that are described in the first embodiment but not described below.

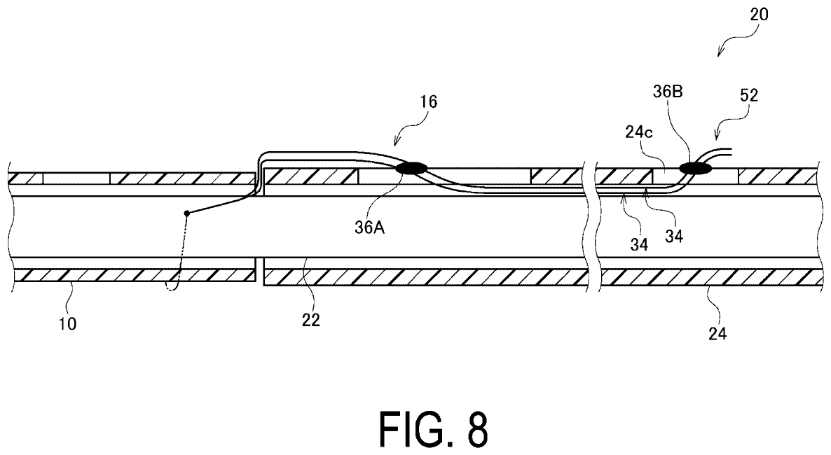

The second embodiment will be described with reference to FIG. 8. The delivery system 20 of the present embodiment differs from the first embodiment in terms of a second coupling part 36B described below. The coupling parts 36A and 36B of the present embodiment include, in addition to the first coupling part 36A, a second coupling part 36B provided at a proximal end side portion of the first thread-like member 16. The second coupling part 36B is provided in the pull-out portion 52 of the first thread-like member 16. The size of the second coupling part 36B is usually larger than the size of the portions of the first thread-like member 16 adjacent to the second coupling part 36B in the length direction. When the portions of the first thread-like member 16 adjacent to the second coupling part 36B in the length direction are aligned on a straight line, the size of the second coupling part 36B referred to here is the maximum outer diameter in a direction orthogonal to a direction along the straight line thereof. By providing such a second coupling part 36B in the pull-out portion 52, the first thread-like member 16 can be easily pinched at a certain location of the second coupling part 36B by the surgeon's fingers, forceps, or the like. Such an effect can be obtained, for example, in a case in which the pull-out portion 52 is pinched in order to eliminate slack of the first outer portion 42 of the first thread-like member 16 when the delivery system 20 is outside the body. In addition, such an effect can also be obtained in a case in which the second coupling part 36B of the first thread-like member 16 is pinched in order to remove the tube stent 10 from within the first organ 12. The size of the second coupling part 36B is set to a size that allows the second coupling part 36B to pass through the first side hole 24b and the second side hole 24c of the outer catheter 24. Accordingly, when the outer catheter 24 is to be separated from the tube stent 10, it is possible to avoid a situation in which the separation of the two is hindered by the second coupling part 36B being caught on either of the side holes 24b, 24c.

The second coupling part 36B of the present embodiment can be disposed in the second side hole 24c of the outer catheter 24. Accordingly, when the delivery system 20 is fed through the lumen of the endoscope, an increase in frictional resistance due to contact between the lumen forming surface of the endoscope and the second coupling part 36B can be suppressed.

### Third Embodiment

The third embodiment will be described with reference to FIG. 9. In the outer catheter 24 of this embodiment, the fourth side hole 24e is positioned further to the distal end side than the first side hole 24b and formed as a side hole other than the first side hole 24b. The first thread-like member 16 of this embodiment is positioned further to the distal end side than the first outer portion 42 and is continuous with the first outer portion 42. The first thread-like member 16 of this embodiment includes a third passing portion 62 that is passed through the fourth side hole 24e. The first outer portion 42 of the first thread-like member 16 differs from that of the first embodiment, and is provided only in a part of the axial range R from the distal end of the outer catheter 24 to the first side hole 24b.

The accommodation hole 60, as in this embodiment, may be formed by the fourth side hole 24e, instead of the first side hole 24b in the first embodiment. The first coupling part 36A that couples the pair of thread-like parts 34 of the first thread-like member 16 may be accommodated in the accommodation hole 60. In this case, the accommodation hole 60, as in this embodiment, may be provided at the distal end of the outer catheter 24. In this case, the accommodation hole 60, as in this embodiment, may open toward the tube stent 10 side in the axial direction.

Next, modified aspects of each of the constituent elements explained above will be described.

The specific mode of non-releasably connecting the first thread-like member 16 to the tube stent 10 is not particularly limited. For example, a terminal end of the first thread-like member 16 may be connected to a part of the tube stent 10 by a knot or the like so as to be non-releasably connected.

Moreover, one of the pair of thread-like parts 34 may form the first outer portion 42 of the first thread-like member 16, and the other thread-like part 34 may be disposed inside the outer catheter 24 in the above-described axial range R. Furthermore, one of the pair of thread-like parts 34 may be disposed inside the tube stent 10, with the other thread-like part 34 not being disposed inside the tube stent 10.

The first thread-like member 16 need not be provided with one or both of the first and second coupling parts 36A and 36B. For example, the first thread-like member 16 may include only the second coupling part 36B.

The accommodation hole 60 need not be an elongated hole. Moreover, the shape of the accommodation hole 60 is not particularly limited, and may be various shapes such as a circular hole, a semicircular hole, or a rectangular hole.

The delivery system 20 need not be provided with the second thread-like member 30. The size relationship between the diameter of the first thread-like member 16 and the diameter of the second thread-like member 30 is not particularly limited, and the sizes of both may be the same. The specific structure for releasably coupling the tube stent 10 and the outer catheter 24 by the second thread-like member 30 is not particularly limited, and various structures including known structures may be adopted. In addition, in the first embodiment, the inner catheter 22 can also be said to form a release member that can be released, through a pulling operation, the connection between the outer catheter 24 and the tube stent 10 by the second thread-like member 30. This release member may be provided separately from the inner catheter 22.

The details of each constituent element described in the above embodiments are merely examples. Technical concepts obtained through abstraction of these details should not be interpreted as limited to the contents of the present specification. Moreover, numerous design changes such as variations, additions, and deletions can be made to the details of the constituent elements described in the embodiments. The details that can be subjected to such design changes are emphasized by adding the notations of the “present embodiment” and “embodiment”. However, design changes are allowed even without such notations. Any combination of the constituent elements described above is also effective. The hatching applied to the cross section of a drawing does not limit the material of the hatched object.

### INDUSTRIAL APPLICABILITY

This disclosure relates to a delivery system.

While preferred embodiments of the disclosure have been described above, it is to be understood that variations and modifications will be apparent to those skilled in the art without departing from the scope and spirit of the disclosure. The scope of the disclosure, therefore, is to be determined solely by the following claims.

1. A delivery system comprising:
an inner catheter;
an outer catheter through which the inner catheter is passed and in which a first side hole is formed;
a tube stent disposed at a distal end side with respect to the outer catheter, the inner catheter being passed through the tube stent; and
a first thread-like member non-releasably connected to the tube stent, at least a portion of the first thread-like member being passed between the inner catheter and the outer catheter at a more proximal end side than the first side hole,
the first thread-like member being passed through the first side hole and being disposed outside the outer catheter in at least a part of an axial range from the distal end of the outer catheter to the first side hole.

2. The delivery system according to claim 1, wherein the first thread-like member includes:
a first passing portion that is passed through the first side hole;
a first inner portion that is continuous with the first passing portion and is passed between the inner catheter and the outer catheter at the more proximal end side than the first side hole; and
a first outer portion that is continuous with the first passing portion and is disposed outside the outer catheter at a more distal end side than the first side hole.

3. The delivery system according to claim 2, wherein the first thread-like member includes a fold-back portion connected to the tube stent, and a pair of thread-like parts connected to the fold-back portion, and
wherein each of the pair of thread-like parts forms the first outer portion.

4. The delivery system according to claim 3, wherein a portion of each of the pair of thread-like parts is disposed inside the tube stent.

5. The delivery system according to claim 2, wherein the first thread-like member includes a fold-back portion connected to the tube stent, and a pair of thread-like parts connected to the fold-back portion, and
wherein the first thread-like member includes a coupling part that couples the pair of thread-like parts.

6. The delivery system according to claim 5, wherein the outer catheter includes an accommodation hole that can accommodate the coupling part and is formed by the first side hole or another side hole formed in the outer catheter at a more distal end side than the first side hole, and
when accommodated in the accommodation hole, the coupling part is provided in a portion that is continuous with an outer portion of the first thread-like member, the outer portion being disposed outside the outer catheter.

7. The delivery system according to claim 6, wherein the accommodation hole is an elongated hole that is long in the axial direction.

8. The delivery system according to claim 2, wherein a second side hole is formed in the outer catheter at a more proximal end side than the first side hole, and
wherein the first thread-like member has a pull-out portion that is pulled out from the second side hole to outside of the outer catheter.

9. The delivery system according to claim 8, wherein the first thread-like member includes a fold-back portion connected to the tube stent, and a pair of thread-like parts connected to the fold-back portion,
wherein the first thread-like member includes a coupling part that couples the pair of thread-like parts, and
wherein the coupling part is provided on the pull-out portion.

10. The delivery system according to claim 2, further comprising a second thread-like member releasably connecting the tube stent and the outer catheter.

11. The delivery system according to claim 10, wherein a diameter of the first thread-like member is larger than a diameter of the second thread-like member.
